(19) United States
(12) Patent Application Publication
(10) Pub. No.: US 2025/0127396 A1
(43) Pub. Date: Apr. 24, 2025

(54) RETINAL CAMERA USING A LIGHT GUIDE TO ILLUMINATE THE EYE WITH A DEFINED LIGHT EMISSION GEOMETRY

(71) Applicant: OPTINA DIAGNOSTICS, INC., Montreal (CA)

(72) Inventors: Jean Philippe SYLVESTRE, Montreal (CA); Antoine DUROCHER-JEAN, Montreal (CA)

(21) Appl. No.: 18/820,658

(22) Filed: Aug. 30, 2024

Related U.S. Application Data
(63) Continuation of application No. PCT/CA2023/050262, filed on Mar. 1, 2023.
(60) Provisional application No. 63/316,061, filed on Mar. 3, 2022.

(51) Int. Cl. A61B 3/15

(52) U.S. Cl. CPC A61B 3/156

(57) ABSTRACT

A retinal camera, comprises a light source, a light guide, optical components and a light sensor. The light source emits light according to a first light emission geometry. The light guide has an illuminated end receiving the light from the light source with a geometry corresponding to the first light emission geometry of the light source. The light source also has a light emitting end receiving the light from the illuminated end and emitting the light according to a second light emission geometry. The optical components direct the light from the light emitting end of the light guide on an illumination path to reproduce the second light emission geometry on an eye of a subject and direct light reflected by the eye of the subject on an imaging path. The light sensor receives the reflected light on the imaging path and creates an image based on the reflected light.

### CROSS-REFERENCE

This application is a continuation of International Patent Application Serial No. PCT/CA2023/050262, filed on Mar. 1, 2023, entitled “RETINAL CAMERA USING A LIGHT GUIDE TO ILLUMINATE THE EYE WITH A DEFINED LIGHT EMISSION GEOMETRY”, which claims priority from U.S. Provisional Patent Application Ser. No. 63/316,061, filed on Mar. 3, 2022, the contents of each of which are incorporated by reference herein in their entirety.

### FIELD

The present technology relates to ophthalmic devices. In particular, the present technology discloses a retinal camera using a light guide to illuminate the eye with a defined light emission.

### BACKGROUND

Retinal cameras have commonly been used to for diagnosis of eye diseases and have more recently been used for diagnosis of other diseases having manifestations in the eye of a subject; see for example International Patent Application Publication No. WO 2018/073784 A1 to Sylvestre et al., published on Apr. 26, 2018, the disclosure of which is incorporated by reference herein in its entirety.

In a retinal camera, also called a fundus camera, illumination and imaging paths typically share a common optical path in order to allow simultaneous illumination and imaging of the retina. As the reflectivity of the retina is small, back reflections from shared optics of the illumination and imaging paths, including the eye itself, may highly deteriorate the quality of captured retinal images. Strategies to overcome this challenge have been developed and typically involve spatially decoupling the areas used for the illumination of the retina and its imaging at the eye pupil plane so to effectively remove back reflections from the cornea of the eye. Typically, an annulus of light is created by inserting a mask in the illumination path in a location optically conjugated with the eye pupil, blocking all the incident light except for an annulus of light. A holed mirror is then placed in a location also optically conjugated with the pupil of the eye, at the intersection of the illumination and imaging paths. This holed mirror reflects the annulus of illumination light towards the eye, while the central, hollow portion of the mirror allows for the reflected light from the fundus through the imaging line to form the image on a sensor. The optical efficacy of such an illumination path is poor as much of the light is blocked by the mask and only a small fraction of the light source passes through the annular-shaped mask. In a typical fundus camera where the light source is a high intensity flash lamp, the mask solution may be adequate since this small fraction is still sufficient to provide adequate illumination of the retina to form a retinal image of adequate quality. However, some other applications rely on light sources having limited power. For example, a laser, a light emitting diode, or the output from a tunable light source may be used in a multispectral or hyperspectral retinal camera, as described in International Patent Application Publication No. WO2016/041062 A1 to Sylvestre et al., published on Mar. 23, 2016, the disclosure of which is incorporated by reference herein in its entirety. In such applications, the cost of a high-power light source may become prohibitive.

FIGS. 1A and 1B (prior art) illustrate variants of conventional fundus camera systems. On FIG. 1A, an imaging system 10 comprises a light source 12, various lenses (or groups of lenses) 14, 16, 18, 20 and 22, a beam splitter 24, a baffle 26 and a camera 28. Light from the light source 12 is projected in an illumination path from the light source 12 through the lenses 14, 16 and the beam splitter 24 onto the pupil of the eye 1 of a subject, allowing the retina to be illuminated. Corneal back reflections are eliminated by placing a central obscuration 30 (annular-shaped mask) in the illumination path conjugate to the pupil of the eye 1. Placement of this central obscuration 30 results in an annular illumination pattern at the pupil of the eye 1. Light reflected by the retina passes on an imaging path through the beam splitter 24, the lenses 18, 20 and 22 and an image of the retina is formed at the camera 28. The baffle 26 is used to reduce corneal back reflections and to limit the entrance pupil diameter of the imaging system 10.

An imaging system 40 shown on FIG. 1B comprises a light source 42, various lenses (or groups of lenses) 44, 46, 48, 50, 52 and 54, a mirror 56 having a central hole and a camera 58. The mirror 56 with the central hole is placed conjugate to the pupil of the eye to combine the illumination and imaging lines and acts as a baffle thereby reducing the corneal back reflections and limiting the entrance pupil diameter of the imaging line. Light from the light source 42 is projected in an illumination path from the light source 42 through the lenses 44, 46, 48, 50, via the mirror 56, and onto the pupil of the eye 1 of a subject allowing the retina to be illuminated. Corneal back reflections are eliminated by placing a central obscuration 60 (annular-shaped mask) in the illumination path conjugate to the pupil of the eye 1. Placement of this central obscuration 60 results in an annular illumination pattern at the pupil of the eye 1. Another central obscuration (or black dot) 62 is placed conjugate to the objective lens 50 and allows to reduce back reflections from the objective lens 50. Light reflected by the retina passes on an imaging path through the lenses 50, 52, 54, via the hole in the mirror 56, and an image of the retina is formed at the camera 58.

Other techniques have been used to form an annulus of light on the eye of the subject. One such technique relies on the use of a conical optical element called an axicon under the condition that incident light on the axicon is of circular geometry to form an annulus of light at the output. However, the tip of the axicon is usually ill-defined, making it quite unusable and thus resulting in undesired losses. Moreover, the use of a diffuser is usually required to achieve a homogeneous illumination of the retina.

Another possible problem encountered in retinal imaging performed with coherent light is often observed in hyperspectral illumination systems with narrow spectral bandwidth. This problem relates to the presence of speckle that degrades the quality of the images that may be captured. Active or passive means may be necessary to reduce the speckle, for example using a moving diffuser to reduce the effect of speckle in an illumination system. This solution also results in optical losses and reduction of the overall efficacy of the optical system.

It is therefore desired to capture as much as possible of light power emitted by a light source. This is of particular importance when the retinal camera operates in multispectral or hyperspectral mode.

Even though the recent developments identified above may provide benefits, improvements are still desirable.

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches.

### SUMMARY

Embodiments of the present technology have been developed based on developers' appreciation of shortcomings associated with the prior art.

In particular, such shortcomings may comprise (1) waste of a large portion of illumination power of light sources; (2) important cost of high-power light sources; and/or (3) presence of speckle degrading the quality of captured retinal images.

In one aspect, various implementations of the present technology provide a retinal camera, comprising: a light source configured to emit light according to a first light emission geometry; a light guide having: an illuminated end configured to receive the light from the light source, the illuminated end having a geometry corresponding to the first light emission geometry of the light source, and a light emitting end configured receive the light from the illuminated end and to emit the light according to a second light emission geometry; at least one first optical component configured to direct the light from the light emitting end of the light guide on an illumination path to reproduce the second light emission geometry on an eye of a subject and to direct light reflected by the eye of the subject on an imaging path; and a light sensor configured to receive the reflected light on the imaging path and to create an image based on the reflected light.

In some implementations of the present technology, the at least one first optical component is configured to replicate the light according to the second light emission geometry on an anterior section of the eye of the subject.

In some implementations of the present technology, the retinal camera further comprises at least one second optical component configured to direct the light from the light source toward the illuminated end of the light guide.

In some implementations of the present technology, the imaging path passes through an aperture formed in the light emitting end of the light guide.

In some implementations of the present technology, the retinal camera further comprises a mask disposed between the light emitting end of the light guide and the at least one first optical component, the mask being configured to block light rays being reflected by the at least one first optical component toward the imaging path.

In some implementations of the present technology, the retinal camera further comprises a mirror having an aperture, the mirror being configured to direct the light from the light emitting end of the light guide toward the at least one first optical component, wherein the imaging path passes through an aperture formed in mirror.

In some implementations of the present technology, the illumination path and the imaging path share a common path between the mirror and the eye of the patient; the mirror is oriented at an angle from the common path; and parts of the illumination path and of the imaging path that are not in the common path are geometrically decoupled.

In some implementations of the present technology, the mirror is oriented at about 45 degrees from the common path; and the parts of the illumination path and of the imaging path that are not in the common path are substantially perpendicular.

In some implementations of the present technology, the retinal camera further comprises at least one third optical component configured to direct the light from the light emitting end of light guide toward the mirror.

In some implementations of the present technology, the retinal camera further comprises a dot positioned on the illumination path, the dot being configured to block light rays being reflected on the illumination path toward the imaging path.

In some implementations of the present technology, the retinal camera further comprises an axicon configured to refract the light on the illumination path and to adjust angles of rays of the light from the light emitting end of the light guide.

In some implementations of the present technology, the retinal camera further comprises an optical power measurement device operatively connected to the light guide.

In some implementations of the present technology, the light guide comprises a light extraction feature configured to direct a minor fraction of the light from the light guide toward the optical power measurement device.

In some implementations of the present technology, the light source is selected from a monochromatic light source and a tunable light source.

In some implementations of the present technology, the second light emission geometry is configured to form an annulus of light on the eye of the subject.

In some implementations of the present technology, the illuminated end of the light guide is configured to match one or more of a shape, a size and an opening angle of the light source.

In some implementations of the present technology, the light guide comprises a first plurality of optical fibers, each optical fiber of the first plurality of optical fibers having a respective input end and a respective output end; the illuminated end of the light guide comprises the input ends of the first plurality of optical fibers, the input ends of the first plurality of optical fibers being assembled according to the first light emission geometry; and the light emitting end of the light guide comprises the output ends of at least a first subset of the first plurality of optical fibers, the output ends of the first subset of the first plurality of optical fibers being assembled according to the second light emission geometry.

In some implementations of the present technology, the output ends of a second subset of the first plurality of optical fibers are spliced away from the output ends of the first subset of the first plurality of optical fibers.

In some implementations of the present technology, the output ends of the second subset of the first plurality of optical fibers are configured to be connected to an optical power measurement device.

In some implementations of the present technology, the light source is a first light source; the retinal camera further comprises a second light source; the light guide further comprises a second plurality of optical fibers, each optical fiber of the second plurality of optical fibers having a respective input end and a respective output end; the illuminated end of the light guide further comprises the input ends of the second plurality of optical fibers, the input ends of the second plurality of optical fibers being assembled according to a light emission geometry of the second light source; the light emitting end of the light guide further comprises the output ends of at least a first subset of the second plurality of optical fibers; and the output ends of the first subset of the first plurality of optical fibers and the output ends of the first subset of the second plurality of optical fibers are assembled according to the second light emission geometry.

In some implementations of the present technology, the output ends of a second subset of the second plurality of optical fibers are spliced away from the output ends of the first subset of the second plurality of optical fibers optical fibers.

In some implementations of the present technology, the output ends of the second subset of the second plurality of optical fibers are configured to be connected to the optical power measurement device.

In some implementations of the present technology, the output ends of the first plurality of optical fibers and the output end of the second plurality of optical fibers are uniformly distributed in the second light emission geometry.

In some implementations of the present technology, N output ends on the second light emission geometry comprises first output ends of N1 optical fibers of the first plurality of optical fibers and second output ends of N2 optical fibers of the second plurality of optical fibers.

In some implementations of the present technology, the light source is a first light source; the retinal camera further comprises a second light source; the illuminated end of the light guide is a first illuminated end; the light guide further comprises a second illuminated end configured to receive light from the second light source, the second illuminated end having a geometry corresponding to a light emission geometry of the second light source; and the light emitting end of the light guide is further configured to combine the light received at the first and second illuminated ends of the light guide.

In some implementations of the present technology, the retinal camera further comprises a memory device; and a controller operatively connected to the memory device, to the light source and to the light sensor, the controller being configured to: cause the light source to emit light toward the illuminated end of the light guide, cause the light sensor to create the image based on the light reflected by the eye of the subject; store the created image in the memory device.

In some implementations of the present technology, the controller comprises a display device, the controller being further configured to cause the display device to display the created image.

In some implementations of the present technology, the at least one first optical component has an adjustable parameter; and the controller is further configured to provide adjustment commands to the at least one first optical component.

In some implementations of the present technology, the light source has an adjustable light emission power, or an adjustable emission wavelength, or both; and the controller is further configured to provide adjustment commands to the light source.

In some implementations of the present technology, the controller is further configured to receive illumination power measurements from the light guide and to provide power adjustment commands to the light source.

In the context of the present specification, unless expressly provided otherwise, a computer system may refer, but is not limited to, an “electronic device”, an “operation system”, a “system”, a “computer-based system”, a “controller unit”, a “monitoring device”, a “control device” and/or any combination thereof appropriate to the relevant task at hand.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives. Still in the context of the present specification, “a” computer-readable medium and “the” computer-readable medium should not be construed as being the same computer-readable medium. To the contrary, and whenever appropriate, “a” computer-readable medium and “the” computer-readable medium may also be construed as a first computer-readable medium and a second computer-readable medium.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIGS. 1A and 1B (prior art) illustrate variants of conventional fundus camera systems;

FIG. 2 is a schematic diagram of a retinal camera in accordance with an embodiment of the present technology;

FIGS. 3A, 3B and 3C illustrate light emission geometries of light sources and illuminated ends of corresponding light guides in accordance with various embodiments of the present technology;

FIG. 4 is a schematic, cross-sectional diagram of a light guide in accordance with an embodiment of the present technology;

FIG. 5 is a schematic diagram of a light guide formed of one or two optical fiber bundles in accordance with an embodiment of the present technology;

FIG. 6 illustrates an annulus of light being projected on the pupil of the eye of a subject in accordance with an embodiment of the present technology;

FIG. 7 is a partial schematic diagram of another retinal camera in accordance with an embodiment of the present technology;

FIG. 8 is a block diagram of a controller of the retinal camera in accordance to an embodiment of the present technology; and

FIG. 9 is a schematic, cross-sectional diagram of a light guide having two illuminated ends in accordance with an embodiment of the present technology.

It should also be noted that, unless otherwise explicitly specified herein, the drawings are not to scale.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements that, although not explicitly described or shown herein, nonetheless embody the principles of the present technology.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes that may be substantially represented in non-transitory computer-readable media and so executed by a computer or processor, whether or not such computer or processor is explicitly shown.

The functions of the various elements shown in the figures, including any functional block labeled as a “processor”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some embodiments of the present technology, the processor may be a general-purpose processor, such as a central processing unit (CPU) or a processor dedicated to a specific purpose, such as a digital signal processor (DSP). Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown. Moreover, it should be understood that module may include for example, but without being limitative, computer program logic, computer program instructions, software, stack, firmware, hardware circuitry or a combination thereof which provides the required capabilities.

In an aspect, the present technology, a retinal camera comprises a light source, a light guide, one or more optical components, and a light sensor. A shape of the light source is such that light is emitted according to a first light emission geometry. The light guide is arranged to capture as much as possible of the light from the light source and to emit the light according to a desired, second light emission geometry, for example and without limitation in the shape of an annulus to be projected on the eye of a subject. To this end, the light guide has an illuminated end having a geometry corresponding to the first light emission geometry of the light source, so that losses of the light emitted by the light source are minimized. The light guide also has a light emitting end receiving the light from the illuminated end and emitting the light according to the desired, second light emission geometry. The one or more optical components direct the light from the light emitting end of the light guide on an illumination path toward the eye of the subject. Light reflected by the eye of the subject, for example by the retina of the subject, is directed by the one or more optical components on an imaging path toward a light sensor. The light sensor, for example a camera, receives the reflected light on the imaging path and creates an image based on the reflected light. The created image may be stored in the camera or in a device connected to the camera, for example a computer or a server.

With these fundamentals in place, we will now consider some non-limiting examples to illustrate various implementations of aspects of the present technology.

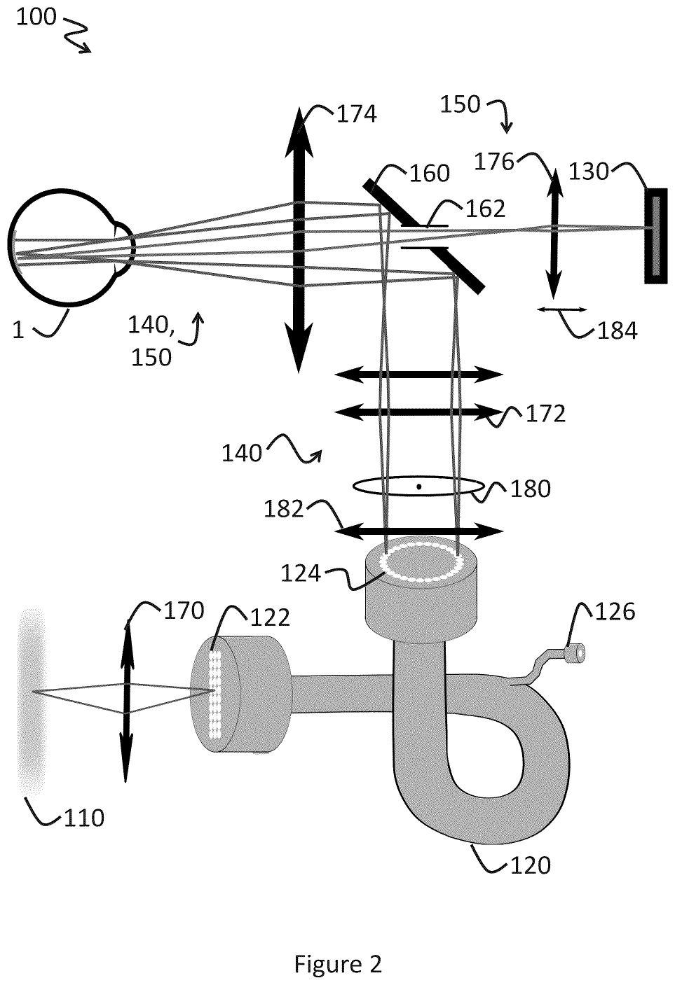

FIG. 2 is a schematic diagram of a retinal camera in accordance with an embodiment of the present technology. A retinal camera 100 usable to obtain an image from an eye 1 of a subject comprises a light source 110, a light guide 120, a light sensor 130 and one or more optical components.

The light guide 120 allows to decouple a geometry of light generation at the light source 110 from a desired illumination geometry on the eye 1 of the subject. The light source 110 may for example be a monochromatic light source and a tunable light source. The light source 110 emits light according to a first light emission geometry. In the non-limiting example of FIG. 2, the first light emission geometry is a rectangle. The light guide 120 has an illuminated end 122 receiving the light from the light source 110. The illuminated end 122 is shaped with a geometry that matches the first light emission geometry of the light source 110, the illuminated end 122 being also rectangular in the non-limiting example of FIG. 2. In more details, the illuminated end 122 of the light guide 120 may match one or more of a shape, a size and an opening angle of the light source 110. The shape of the illuminated end 122 matches the shape of the light emission by the light source 110 in order to maximize the capture of light by the light guide 120 and to minimize losses of the light emitted by the light source 110. The light guide 120 also has a light emitting end 124 that receives the light from the illuminated end 122 and emits the light according to a second light emission geometry. In the non-limiting example of FIG. 2, the second light emission geometry is an annulus (or ring) so that the retinal camera 100 may form an annulus of light on the eye 1 of the subject.

At least one optical component, actually including more than one optical components in the non-limiting example of FIG. 2, direct the light from the light emitting end 124 of the light guide 122 on an illumination path 140 toward the eye 1 of the subject and direct light reflected by the eye 1 of the subject on an imaging path 150. The light sensor 130, for example a camera, receives the reflected light on the imaging path 150 and creates an image based on the reflected light. As illustrated, the optical components include reflective optics, for example a mirror 160 having an aperture 162. The light emitted on the illumination path 140 by the light emitting end 124 of the light guide 120 is reflected by the mirror 160 and redirected toward the eye 1 of the subject. In this manner, the mirror 160 replicates the light emitted according to the second light emission geometry by the light emitting end 124 of the light guide on an anterior section of the eye 1 of the subject, for example on the plane of the eye pupil. The light reflected into the imaging path 150 by the eye 1 of the subject passes through the aperture 162 of the mirror 160 and continues on the imaging path 150 until it reaches the light sensor 130.

Other optical components that may be present in various embodiments include lenses (or groups of lenses) 170, 172, 174, 176, a dot 180, and an axicon lens 182. As illustrated, the lens 170 is used to direct and focus the light from the light source 110 on the illuminated end 122 of the light guide 120. The lens 170 may be omitted, for example and without limitation when the light source 110 is very close to the illuminated end 122 of the light guide 120. The axicon lens 182 is positioned very close to the light emitting end 124 of the light guide 120 (with or without the use of a matching index material between the light guide 120 and the axicon lens 182) and is used to refract the light on the illumination path 140 and to adjust angles of rays of the light from the light emitting end 124 of the light guide 120 on the illumination path 140 in order to optimize the transmission efficacy of the illumination path 140 and/or the illumination uniformity on the retina. Another lens 174 is present on a common path of the illumination and imaging paths 140, 150 between the eye 1 and the mirror 160. The lens 174 focuses light in both directions between the eye 1 and the mirror 160. The mirror 160 may be oriented at an angle from the common path, in order to decouple parts of the illumination path 140 and imaging path 150 that are not in the common path. As illustrated, the angle of the mirror 160 is about 45 degrees from the common path and the parts of the illumination and imaging paths 140, 150 that are not in the common path are substantially perpendicular. Other angular configurations are also contemplated.

The one or more lenses 172 direct the light from the emitting end 124 of the light guide 120 toward the mirror 160 and allow for the introduction of the dot 180 conjugated to the lens 174 and used to block light rays being reflected on the illumination path 140 toward the imaging path 150. The lens 176 is positioned on the imaging path 150 before the light sensor 130 and is adjustable along a direction 184 so that an image can be formed on the light sensor 130 while accommodating a refractive error on the eye 1 of the subject.

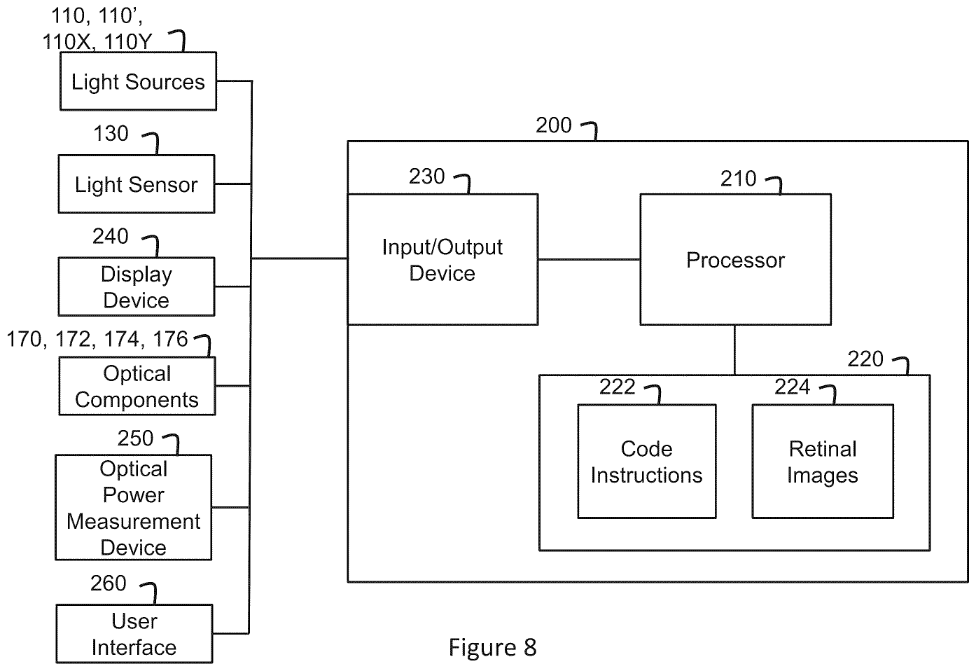

In an embodiment, the light guide 120 comprises a light extraction feature 126 that directs a minor fraction of the light from the light guide 120 toward the optical power measurement device (FIG. 8) used to monitor the operation of the retinal camera 100.

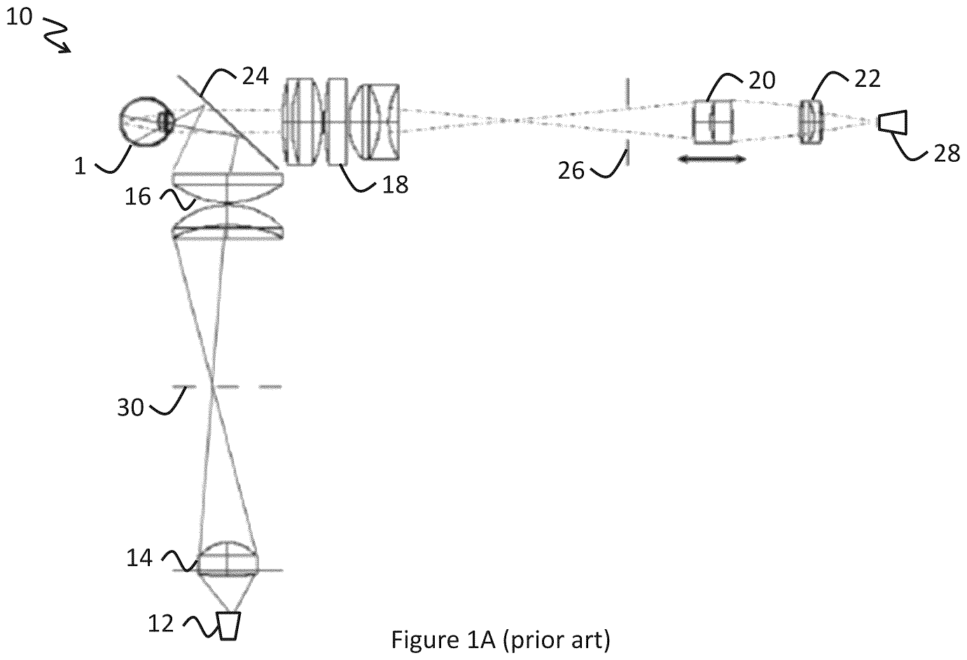

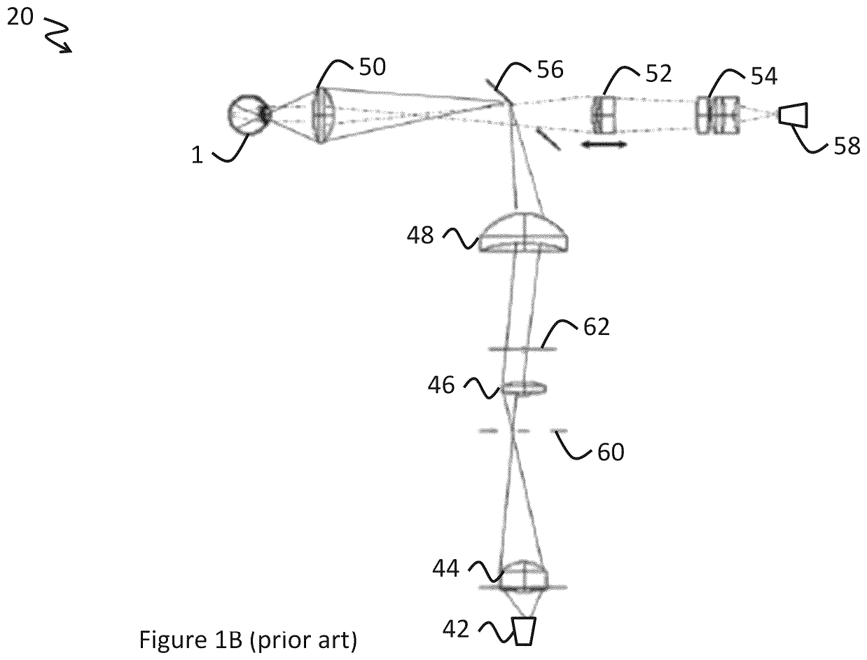

In an embodiment, a conventional retinal camera may be modified by replacing some optical components present in its illumination path, for example the optical components to form the annulus of light based on a light source 12, an optical component 14 and an annular-shaped mask 30 from FIG. 1 a) or their corresponding elements 42, 44 and 60 from FIG. 1 b), with the light emitting end 124 of the light guide 120. The light emitting end 124 of the light guide 120 may be positioned at the same location as the annular-shaped mask 30 (FIG. 1A) or 60 (FIG. 1B) it is replacing, may have the same geometry, and may reproduce the same angular distribution of light.

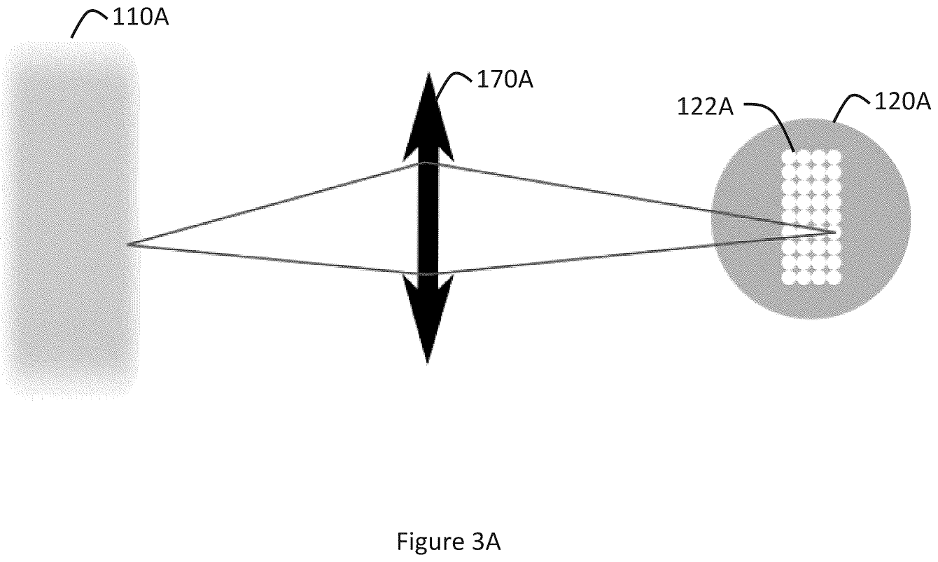

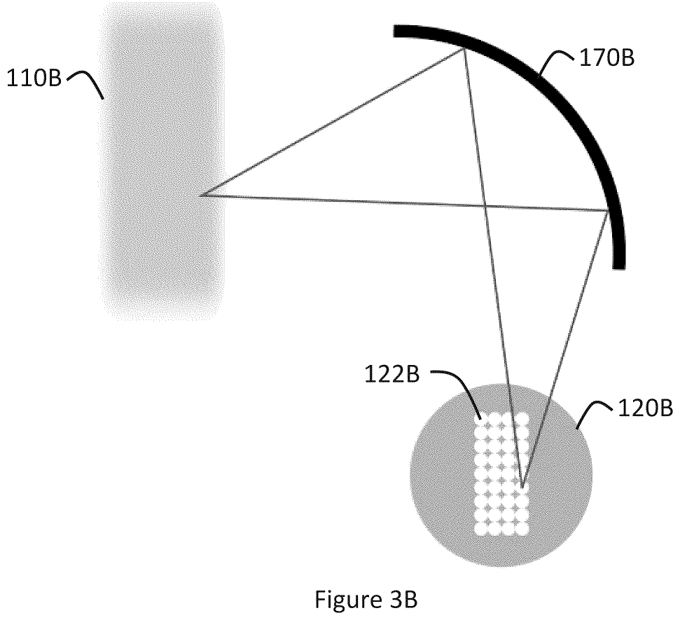

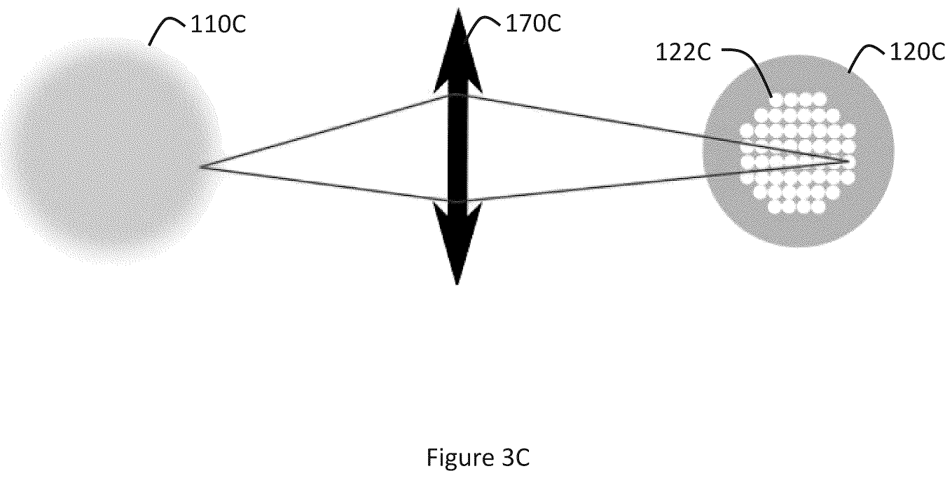

FIGS. 3A, 3B and 3C illustrate light emission geometries of light sources and illuminated ends of corresponding light guides in accordance with various embodiments of the present technology. On FIG. 3A, a light source 110A emits light according to a rectangular geometry. The light source 110A may for example be a discharge lamp such as an arc lamp, a hollow cathode lamp, a neon tube, an incandescent lamp. The light source may also be any broadband light source spectrally filtered with a rectangular output geometry, such as a tunable light source with Bragg grating, with a monochromator having a slit, or with a Fabry-Pérot interferometer. Rectangular or square light emission geometries may also be produced by a matrix formed of a plurality of light emitting diodes (LED). A light guide 120A has an illuminated end 122A defining the same rectangular geometry. Refractive optics such as a lens (or a group of lenses) 170A directs and focuses the light from the light source 110A on the illuminated end 122A of the light guide 120A, the directed light substantially meeting a shape, size and angular dispersion of the illuminated end 122A of the light guide 120A.

On FIG. 3B, a light source 110B also emits light according to a rectangular geometry. A light guide 120B has an illuminated end 122B defining the same rectangular geometry. Reflective optics such as a mirror 170B, which may be flat or curved, is used alone or in combination with additional optical components (not shown) to direct and focus the light from the light source 110B on the illuminated end 122B of the light guide 120B, the directed light substantially meeting a shape, size and angular dispersion of the illuminated end 122B of the light guide 120B.

On FIG. 3C, a light source 110C emits light according to a circular geometry. The light source 110B may for example be a laser light source, or comprise fibered laser light sources (e.g. supercontinuum, spectrally filtered or not), or be provided as incandescent lamps or light bulbs in general, with or without a parabolic reflector. A light guide 120C has an illuminated end 122C defining the same circular geometry. A lens (or a group of lenses) 170C directs and focusses the light from the light source 110C on the illuminated end 122C of the light guide 120C, the directed light substantially meeting a shape, size and angular dispersion of the illuminated end 122C of the light guide 120C. Other light emission geometries and other combinations of optical elements between the light sources and the light guides are also contemplated, so the examples of FIGS. 3A, 3B and 3C are not intended to limit the scope of the present disclosure. For example, the mirror 170B may be used to direct and focus the light emitted with a circular geometry by light source 110C.

It should be understood that the present technology is not limited by the construction or type of the light source 110A, 110B or 110C.

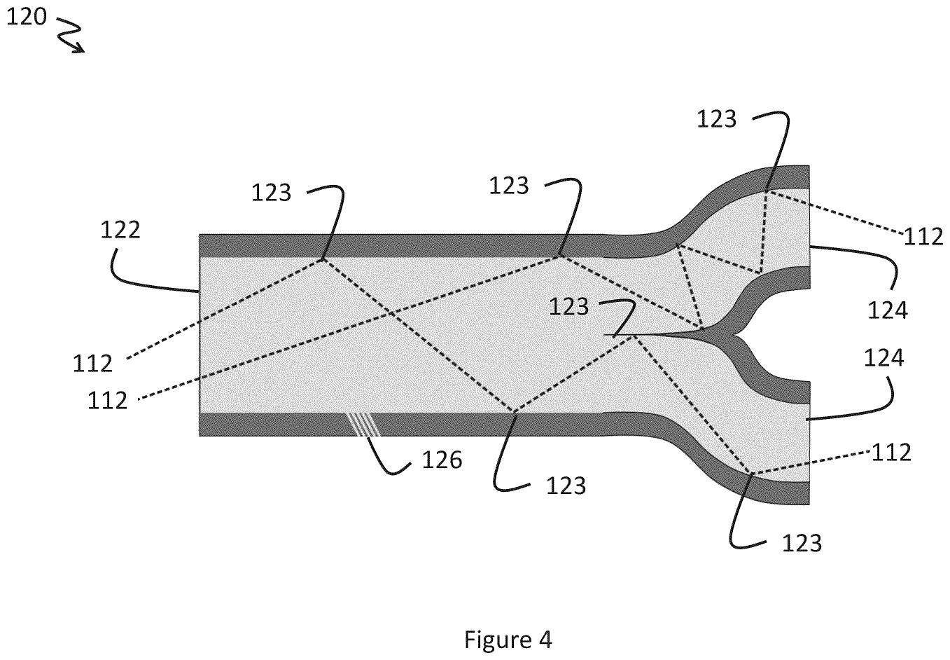

In at least one embodiment, the light guide 120 may be constructed of a single piece of a transparent material, such as plastic or glass, with a refractive index of about 1.40 to 1.60 and a good optical transmission in the spectral range of the light to be delivered in the retinal camera, for example covering the visible and near-infrared portion of the spectrum. FIG. 4 is a schematic, cross sectional diagram of a light guide 120 in accordance with an embodiment of the present technology. Light rays 112 received at the illuminated end 122 are conducted via successive areas 123 of internal reflections, with minimal losses, up to the light emitting end 124. In an embodiment, loss of light within the light guide 120 is minimized by maintaining a substantially constant cross-section of the light guide 120 from the illuminated end 122 to the emitting end 124. A small fraction of the light is being conducted out to the light extraction feature 126. This light extraction feature 126 may consist in small, prism-like structures cut into the guide or dots of paint applied onto the surface that are locally adding texture to the light guide, breaking the condition for total internal reflection and therefore allowing for extracting a fraction of the light from the light guide 120.

In at least one other embodiment, the light guide 120 may be constructed as a bundle containing a plurality of optical fibers, each optical fiber having a respective input end and a respective output end. The illuminated end 122 of the light guide 120 may be formed by assembling the input ends of the optical fibers according to the first light emission geometry for matching the light emitted by the light source 110. The input ends of the optical fibers may be fused in order to improve the efficiency of the light collection by reducing free spaces between the optical fibers. The light emitting end 124 of the light guide 120 may then be formed by assembling the output ends of at least a large subset of the plurality of optical fibers, the output ends of the subset of the plurality of optical fibers being assembled according to the second light emission geometry. The output ends of a smaller subset of the plurality of optical fibers may be spliced away from the output ends of the larger subset of the plurality of optical fibers to form the light extraction feature 126, which may be connected to an optical power measurement device.

A number of optical fibers in the bundle, as well as their core diameters and numerical apertures of the optical fibers, may be selected in view of directing a major fraction of the light from the light source 110 on the eye 1 of the subject and in view of providing a uniform illumination of the retina 1 according to a specific pattern. For example, the fibers may be step-index or graded-index multimodal with a core diameter of 10 to 2000 micrometers and a numerical aperture of 0.10 to 0.55. The light guide 120 (bundle) directs the light from the light source to the eye 1 of the subject via the one or more optical component, for example the lens 172, the mirror 160 and the lens 174, illuminates the retina by entering the eye 1 in the second light emission geometry, directly allowing a decoupling of the incoming light on the illumination path 140 into the eye 1 from the reflected light exiting the eye 1 on the imaging path 150. The light according to the second light emission geometry, for example the annulus or light, is formed in the anterior portion of the eye 1 (e.g. on the pupil of the eye).

In an embodiment, an angle of each individual fiber relative to an optical axis of the illumination path 140 may be adjusted to direct the light in a manner that optimizes uniformity of the illumination on the retina. It may be noted that the use of multiple fibers to illuminate the retina of the eye 1 of the subject may reduce the presence of speckle on the resulting retinal image, as each point of the retina receives light from multiple individual fibers. Consequently, the need to resort to other speckle reducing devices may be avoided.

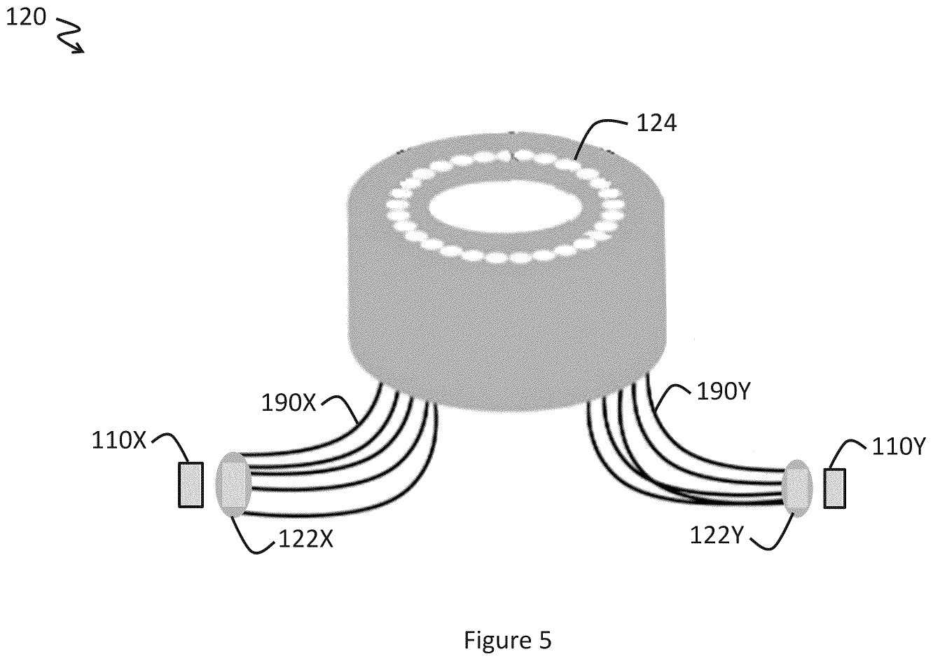

In an embodiment, the retinal camera 100 may comprise a second light source and the light guide 120 may comprise a second plurality of optical fibers. FIG. 5 is a schematic diagram of a light guide formed of one or two optical fiber bundles in accordance with an embodiment of the present technology. The light guide 120 may comprise a first illuminated end 122X receiving light from a first light source 110X and may optionally comprise a second illuminated end 122Y receiving light from a second light source 110Y. A first bundle comprising a first plurality optical fibers 190X directs light from the first illuminated end 122X to the light emitting end 124. A second bundle comprising a second plurality of optical fibers 190X, if present, directs light from the second illuminated end 122Y to the light emitting end 124. Each optical fiber of these first and second pluralities of optical fibers 190X, 190Y may also have a respective input end and a respective output end. The input ends of the optical fibers of the second plurality are assembled according to a light emission geometry of the second light source 110Y, which may be similar to, or different from, the light emission geometry of the light source 110X. Otherwise stated, the first light source 110X and the second light source 110Y may have the same or different constructions that provide different intensities and/or wavelength ranges. In this embodiment, the light emitting end 124 of the light guide 120 may at once comprises the output ends of at least a large subset of the output ends of the first and second pluralities of optical fibers, these output ends being assembled according to the second light emission geometry. The output ends of a small subset of the second plurality of optical fibers 190Y may also be spliced away from the other output ends of the second plurality of optical fibers optical fibers in order to form another light extraction feature (not shown) connectable to the same or another optical power measurement device. Defining more pluralities of optical fibers in the light guide 120 for collecting light from additional light sources is also contemplated.

In an embodiment, each group of N output ends on the second light emission geometry comprises first output ends of N1 optical fibers of the first plurality of optical fibers 190X and second output ends of N2 optical fibers of the second plurality of optical fibers 190Y, in which N is equal to N1 plus N2, both of N1 and N2 equal to N/2 in a non-limiting example. The output ends of the first plurality of optical fibers 190X and the output end of the second plurality of optical fibers 190Y may be uniformly distributed in the second light emission geometry. For example, each pair of adjacent output ends of the second light emission geometry may comprise one output end of the first plurality of optical fibers 190X projecting light from the first light source 110X and one output end of the second plurality of optical fibers 190Y projecting light from the second light source 110Y. These configurations may allow, for example, selecting to illuminate the eye 1 of the subject using light from either one or from both of the first and second light sources 110X, 110Y while maintaining the same second light emission geometry at the illuminating end 124 of the light guide 120. Assembling the output ends of the second plurality of optical fibers to form a different light emission geometry at the illuminating end 124 is also contemplated.

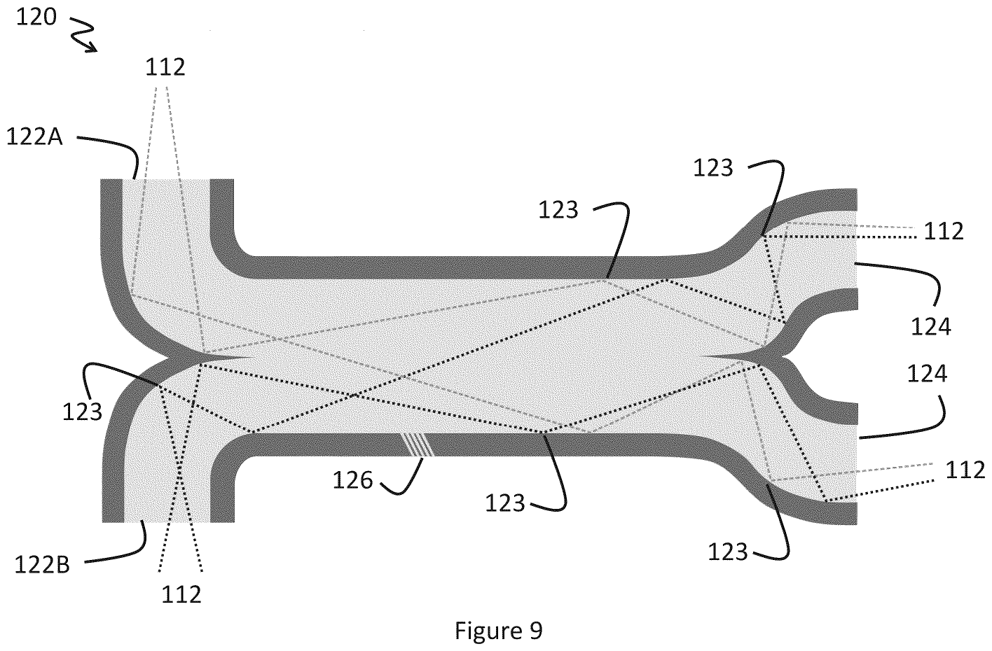

FIG. 9 is a schematic, cross-sectional diagram of a light guide having two illuminated ends in accordance with an embodiment of the present technology. A light guide 120 differs from the light guide 120 introduced in FIG. 4 in that it comprises two illuminated ends 122A and 122B that are each configured to receive light from distinct light sources, for example from the light sources 110X and 110Y (FIG. 5). Other than the presence of two distinct illuminated ends 122A and 122B, the light guide shown in FIG. 9 may be constructed in the same manner as that of FIG. 4 and may be used, for example, for selecting to illuminate the eye 1 of the subject using light from either one or from both of the first and second light sources 110X, 110Y while maintaining the same second light emission geometry at the illuminating end 124 of the light guide 120.

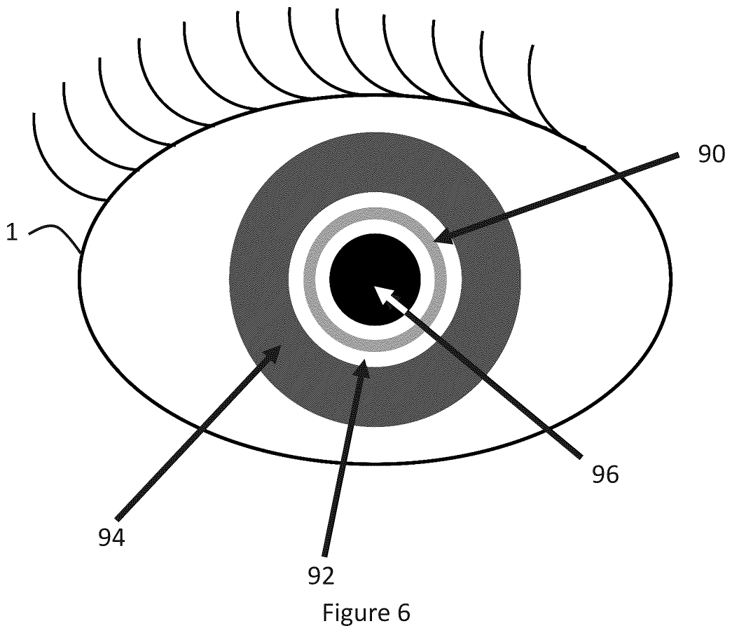

As mentioned hereinabove, the second light emission geometry formed by the light emitting end 124 of the light guide 120 may have various shapes selected according to the needs of a particular application. One such application defines the second light emission geometry as an annulus, or ring, for projection on the eye 1 of the subject. FIG. 6 illustrates an annulus of light being projected on the pupil of the eye of a subject in accordance with an embodiment of the present technology. An annulus of light 90 is formed on the pupil 92 of the eye 1 of the subject. The various components of the retinal camera 100, including the light guide 120 and some of the optical components, are configured such that annulus of light 90 is smaller than an opening of the iris 94. In an embodiment, the configuration of the retinal camera 100 is such that the annulus of light 90 is smaller than the opening of the iris 94 in the context of non-mydriatic imaging, so that chemically-induced dilation of the pupil 92 is not required. Light reflected by the retina of the eye 1 of the subject passed through the pupil 92 within an imaging disk area 96 that is smaller than an opening of the annulus of light 90. The annulus of light 90 reproduces (is being conjugated with) the geometry of the light emitting end 124 of the light guide 120 while an area of capture of the light sensor 130 is conjugated with the imaging disk area 96, in view of preventing overlap between light present in the illumination and imaging paths 140, 150 and in view of minimizing unwanted artefacts, such as parasitic reflections, in the retinal image formed by the light sensor 130.

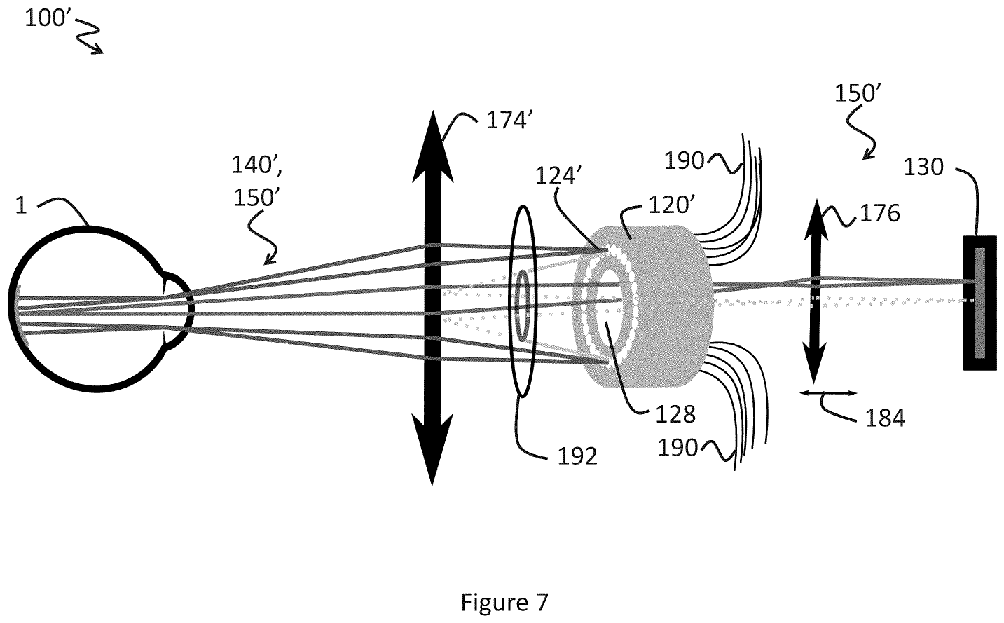

FIG. 7 is a partial schematic diagram of another retinal camera in accordance with an embodiment of the present technology. In a retinal camera 100′, light from a light source (not shown but for example equivalent to the light source 110 of FIG. 2) is transmitted within a light guide 120′ (only a part of which being shown) from an illuminated end (not shown) to a light emitting end 124′ that may share the characteristics of the light emitting end 124 of the light guide 120 introduced hereinabove. In more details, the light is transmitted within the light guide 120′ via a plurality of optical fibers 190. The light from the light emitting end 124′ of the light guide 120′ is directed toward the eye 1 of the subject on an illumination path 140′ via one or more optical elements, for example via a lens (or a group of lenses) 174′. Light reflected on an imaging path 150′ by the eye 1 of the subject passes through the lens 174′ and through an aperture 128 formed within a perimeter of the light emitting end 124′ of the light guide 120′ and reaches the camera 130. In an embodiment, a mask 192 may be disposed on the imaging path 150′, for example between the lens 174′ and the light emitting end 124′ of the light guide 120′. The mask 192 allows to block light rays being reflected by lens 174′ or any other optical component toward the imaging path 150′. The lens 176 may also be present on the imaging path 150′ and may also be moved along the direction 184 so that an image can be formed on the light sensor 130 while accommodating a refractive error on the eye 1 of the subject. Although not shown on FIG. 7, the light guide 120′ may include a light extraction feature equivalent to the light extraction feature 126 illustrated on FIG. 2. Generally speaking, the retinal cameras 100 and 100′ may be used in the same manner in view of obtaining retinal images of equivalent quality.

FIG. 8 is a block diagram of a controller of the retinal camera in accordance to an embodiment of the present technology. On FIG. 8, a controller 200 includes a processor or a plurality of cooperating processors (represented as one processor 210 for simplicity), a memory device or a plurality of memory devices (represented as one memory device 220 for simplicity), an input/output device or a plurality of input/output devices (represented as one input/output device 230 for simplicity). Distinct input and output devices may be implemented instead of the input/output device 230. The processor 210 is operatively connected to the memory device 220 and to the input/output device 230. The memory device 220 may comprise a non-transitory computer-readable media 222 for storing code instructions that are executable by the processor 210 for controlling the retinal camera 100 or 100′. The memory device 220 may comprise a repository 224 for storing retinal images created by the retinal camera 100 or 100′.

In an embodiment, the controller 200 may be communicatively connected, via the input/output device 230, to the light source 110 or 110′ and to any additional second light source 110X and/or 110Y, to the light sensor 130, to one or more of the optical components 170, 172, 174 and to any other optical component, to a display device 240, to one or more optical power measurement devices 250, and to a user interface 260. The controller 200 may receive retinal image data created by the light sensor 130 and cause the display device 240 to display the retinal image. The controller 200 may also store the retinal image in the repository 224 of the memory device 220. Information related to the subject for whom the retinal image is being created may be received from the user interface 260 and shown on the display device 240 or stored in the repository 224.

Commands may be received at the controller 200 from the user interface 260 for causing one or more adjustments of the retinal camera 100 or 100′. Data related to these commands may be stored in the memory device 220 so that such adjustments may be maintained until they are updated again by an operator. Such commands may be translated by the processor 210 such that corresponding commands are sent, via the input/output device 230 to light source 110, 110′ for adjusting one or both of a light emission power and a light emission wavelength. A feedback loop may be implemented in the controller 200 by using measurements from the light guide 120 or 120′ obtained by the optical power measurement device 260 in order to control the light emission power of the light source 110, 110′. These light power measurements may also be reported on the display device 240. Other commands resulting from interactions with the user interface 260 may be sent to the one or more optical components 170, 172, 174, 176 for adjusting its parameters, for example for adjusting a focus on the illumination path 140 and/or for accommodating a refractive error on the eye 1 of the subject on the imaging path 150. In some embodiments, the controller 200 may adjust a position of the retinal camera 100 or 100′ so that the annulus of light 90 properly reaches the pupil of the subject. Adjusting an internal fixation light is also contemplated

If the retinal camera 100 or 100′ includes more than one light source 110 or 110′ and more than one corresponding light guide 120 or 120′, the controller 200 may selectively turn on one or both of the light sources, for example according to commands received on the user interface 260, for obtaining distinct images of the retina of the eye 1 of the patient.

While the above-described implementations have been described and shown with reference to particular steps performed in a particular order, it will be understood that these steps may be combined, sub-divided, or re-ordered without departing from the teachings of the present technology. At least some of the steps may be executed in parallel or in series. Accordingly, the order and grouping of the steps is not a limitation of the present technology.

It should be expressly understood that not all technical effects mentioned herein need to be enjoyed in each and every embodiment of the present technology.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1. A retinal camera, comprising:
a light source configured to emit light according to a first light emission geometry;
a light guide having:
an illuminated end configured to receive the light from the light source, the illuminated end having a geometry corresponding to the first light emission geometry of the light source, and
a light emitting end configured receive the light from the illuminated end and to emit the light according to a second light emission geometry;
at least one first optical component configured to direct the light from the light emitting end of the light guide on an illumination path to reproduce the second light emission geometry on an eye of a subject and to direct light reflected by the eye of the subject on an imaging path;
an axicon configured to refract the light on the illumination path and to adjust angles of rays of the light from the light emitting end of the light guide;
and
a light sensor configured to receive the reflected light on the imaging path and to create an image based on the reflected light.

2. The retinal camera of claim 1, wherein the at least one first optical component is configured to replicate the light according to the second light emission geometry on an anterior section of the eye of the subject.

3. The retinal camera of claim 1, further comprising at least one second optical component configured to direct the light from the light source toward the illuminated end of the light guide.

4. The retinal camera of claim 1, wherein the imaging path passes through an aperture formed in the light emitting end of the light guide.

5. The retinal camera of claim 4, further comprising a mask disposed between the light emitting end of the light guide and the at least one first optical component, the mask being configured to block light rays being reflected by the at least one first optical component toward the imaging path.

6. The retinal camera of claim 1, further comprising a mirror having an aperture, the mirror being configured to direct the light from the light emitting end of the light guide toward the at least one first optical component, wherein the imaging path passes through an aperture formed in mirror.

7. The retinal camera of claim 6, wherein:
the illumination path and the imaging path share a common path between the mirror and the eye of the patient;
the mirror is oriented at an angle from the common path; and
parts of the illumination path and of the imaging path that are not in the common path are geometrically decoupled.

8. The retinal camera of claim 7, wherein:
the mirror is oriented at about 45 degrees from the common path; and
the parts of the illumination path and of the imaging path that are not in the common path are substantially perpendicular.

9. The retinal camera of claim 6, further comprising at least one third optical component configured to direct the light from the light emitting end of light guide toward the mirror.

10. The retinal camera of claim 9, further comprising a dot positioned on the illumination path, the dot being configured to block light rays being reflected on the illumination path toward the imaging path.

11. (canceled)

12. The retinal camera of claim 1, further comprising an optical power measurement device operatively connected to the light guide.

13. The retinal camera of claim 12, wherein the light guide comprises a light extraction feature configured to direct a minor fraction of the light from the light guide toward the optical power measurement device.

14. (canceled)

15. (canceled)

16. (canceled)

17. The retinal camera of claim 1, wherein:
the light guide comprises a first plurality of optical fibers, each optical fiber of the first plurality of optical fibers having a respective input end and a respective output end;
the illuminated end of the light guide comprises the input ends of the first plurality of optical fibers, the input ends of the first plurality of optical fibers being assembled according to the first light emission geometry; and
the light emitting end of the light guide comprises the output ends of at least a first subset of the first plurality of optical fibers, the output ends of the first subset of the first plurality of optical fibers being assembled according to the second light emission geometry.

18. The retinal camera of claim 17, wherein the output ends of a second subset of the first plurality of optical fibers are spliced away from the output ends of the first subset of the first plurality of optical fibers.

19. The retinal camera of claim 18, wherein the output ends of the second subset of the first plurality of optical fibers are configured to be connected to an optical power measurement device.

20. The retinal camera of claim 17, wherein:
the light source is a first light source;
the retinal camera further comprises a second light source;
the light guide further comprises a second plurality of optical fibers, each optical fiber of the second plurality of optical fibers having a respective input end and a respective output end;
the illuminated end of the light guide further comprises the input ends of the second plurality of optical fibers, the input ends of the second plurality of optical fibers being assembled according to a light emission geometry of the second light source;
the light emitting end of the light guide further comprises the output ends of at least a first subset of the second plurality of optical fibers; and
the output ends of the first subset of the first plurality of optical fibers and the output ends of the first subset of the second plurality of optical fibers are assembled according to the second light emission geometry.

21. (canceled)

22. (canceled)

23. The retinal camera of claim 1, wherein the output ends of the first plurality of optical fibers and the output end of the second plurality of optical fibers are uniformly distributed in the second light emission geometry.

24. The retinal camera of claim 1, wherein N output ends on the second light emission geometry comprises first output ends of N1 optical fibers of the first plurality of optical fibers and second output ends of N2 optical fibers of the second plurality of optical fibers.

25. The retinal camera of claim 1, wherein:
the light source is a first light source;
the retinal camera further comprises a second light source;
the illuminated end of the light guide is a first illuminated end;
the light guide further comprises a second illuminated end configured to receive light from the second light source, the second illuminated end having a geometry corresponding to a light emission geometry of the second light source; and
the light emitting end of the light guide is further configured to combine the light received at the first and second illuminated ends of the light guide.

26. The retinal camera of claim 1, further comprising:
a memory device; and
a controller operatively connected to the memory device, to the light source and to the light sensor, the controller being configured to:
cause the light source to emit light toward the illuminated end of the light guide,
cause the light sensor to create the image based on the light reflected by the eye of the subject;
store the created image in the memory device.

27. (canceled)

28. (canceled)

29. (canceled)

30. (canceled)
